(19) United States
(12) Patent Application Publication
(10) Pub. No.: US 2002/0156084 A1
(43) Pub. Date: Oct. 24, 2002

(54) Use of N-benzyl piperazine and analogues as quality of life enhancers

(76) Inventor: Bryant G. Valent, Naples, FL (US)

Correspondence Address: Bryant G. Valent, 101 Plam Frond Ct, Naples, FL, 34104, US

(21) Appl. No.: 10/113,993

(22) Filed: Apr. 1, 2002

Related U.S. Application Data
(60) Provisional application No. 60/281,208, filed on Apr. 3, 2001.

(51) Int. Cl. A61K 31/495; A61K 31/496; C 07D 4 5/02

(52) U.S. Cl. 514/252.12; 514/254.11; 544/378; 544/403

(57) ABSTRACT

A chemical composition, namely n-benzyl piperazine and derivatives, for use as a weight control product and or a quality of life enhancer. The unobviousness lies in the fact that the aforementioned compounds work without the unwanted side effects of many weight control products, due to their “harmonious” balancing of the norepinephrine and serotonin receptors in the brain.

### DESCRIPTION

This Patent description is of the use of N-Benzyl Piperazine and Analogues as Quality of Life Enhancers. Administration of N-Benzyl Piperazine has shown to be effective as an appetite suppressant and as a mild anti-depressant. Yeah though it be known that N-Benzyl Piperazine is a CNS stimulant and that stimulants usually are appetite suppressants, it was unknown and unobvious that the binding affinities for the serotonin receptor would be in “perfect” harmony with the norepinephrine receptor to eliminate the side effects of nervousness and the like. It was also unobvious that the increased levels of serotonin and norepinephrine would facilitate individuals in their daily lives to accomplish goals. That is why it is referred to as a Quality of Life Enhancer. Quality of Life Enhancer is defined as a weight loss supplement, appetite suppressant, anti-depressant, cognition enhancer or diet supplement.

We also propose that it is unobvious that co-administration of a serotonin agonist or re-uptake blocker, would have such a profound effect on the undesirable side effects of current CNS stimulant weight loss products.

### PROCEDURE

A solution of 24.3 g. (0.125 mole) of piperazine hexahydrate in 50 ml. of absolute ethanol, contained in a 250-ml Erlenmeyer flask, is warmed in a bath at 65° C. as there is dissolved in the solution, by swirling, 22.1 g. (0.125 mole) of piperazine dihydrochloride monohydrate. As warming in the bath at 65° C. is continued, there is added during 5 minutes, with vigorous swirling or stirring, 15.8 g (14.3 ml, 0.125 mole) of recently distilled benzyl chloride. The separation of white needles comes almost immediately. After the solution has been stirred for an additional 25 minutes at 65° C., it is cooled, and the unstirred solution is kept in an ice bath for about 30 minutes. The crystals of piperazine dihydrochloride monohydrate are collected by suction filtration, washed with three 10 ml portions of ice-cold absolute ethanol, and then dried. Recovery of the dihydrochloride is 21.5-22.0 g. (97-99%).

The combined filtrate and washings from the piperazine dihydrochloride are cooled in an ice bath and treated with 25 ml of absolute ethanol saturated at 0° C. with dry hydrogen chloride . After the solution has been well mixed, it is cooled for 10-15 minutes in an ice bath. The precipitated white plates of 1-benzylpiperazine dihydrochloride are collected by suction filtration, washed with dry benzene, and dried. The product, which melts at about 280° C. with decomposition, after sintering at about 254° C., amounts to 29-29.5 g. (93-95%). A solution of this salt in 50 ml of water is made alkaline (pH>12) with about 60 ml of 5N sodium hydroxide, then extracted twelve times with 20 ml portions of chloroform. The combined extracts are dried over anhydrous sodium sulfate, and the pale brown oil remaining after removal of solvent is distilled at reduced pres in a Claisen flask. The yield of pure 1-benzylpiperazine, bp 122-124° C./2.5 mmHg, is 14.3-16.5 g. (65-75%).

### Method Of Use

Administration of the compounds is usually in the form of their pharmaceutically acceptable acid addition salts. Doses are within the range if 10 mg to 300 mg per day, but usually are in pills of 75 mg two times a day. Once desired effect is attained 75 mg per day is sufficient.

What is claimed: A method of enhancing the quality of life by administration of:

1. A. administration of N-benzyl Piperazine in an amount to cause said effect.
B. N-Benzyl Piperazine as the free base or a pharmaceutically acceptable acid addition salt.

2. -(Phenylethyl)-piperazine

3. A compound of the following formula where R1 and R2 are from the following group: lower alkyl, amino, halo, methylenedioxy, carbonyl, phenyl, trifluoromethyl, methoxy, ethoxy. And n is an integer from 1 to 10.

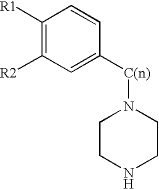

4. Where said method of enhancing quality of life is referred to as
A. Dietary Aid
B. Dietary Supplement
C. Appetite Suppressant
D. Cognition Enhancer
E. Fat Loss Catalyst
F. Weight Loss Supplement
G. Quality of Life Enhancer

5. Administration of a CNS stimulant in conjunction with
A. Serotonin Agonist
B. Serotonin reuptake blocker to cause said effect of quality of life enhancement.
